(19) United States
(12) Patent Application Publication
(10) Pub. No.: US 2010/0249611 A1
(43) Pub. Date: Sep. 30, 2010

(54) Respiratory Signal Processing Method

(75) Inventors: Liu Chen, Nanshan Shenzhen (CN); Min Wang, Nanshan Shenzhen (CN); Hongchun Wang, Nanshan Shenzhen (CN)

Correspondence Address: GARCIA-ZAMOR INTELLECTUAL PROPERTY LAW;ATTN: RUY GARCIA-ZAMOR, 12960 LINDEN CHURCH ROAD, CLARKSVILLE, MD, 21029, US

(73) Assignee: Edan Instruments. Inc., Shekou (CN)

(21) Appl. No.: 12/470,304

(22) Filed: May 21, 2009

(30) Foreign Application Priority Data
Mar. 26, 2009 (CN) 200910106234.3

(51) Int. Cl. A61B 5/0205; A61B 5/08

(52) U.S. Cl. 600/484; 600/529

(57) ABSTRACT

This Invention relates to a respiratory signal processing method, including the following steps: A. Obtain the respiratory physiological signals by means of a specific respiratory circuit, AD collects and obtains the respiratory digital signals and applies the data preprocessing to such signals; B. Convert frequency spectrum of the preprocessed respiratory signals and obtain the distribution of their frequency domain; C. Judge whether there is asphyxia in accordance with the properties of respiratory signal's physiological parameters; D. Introduce the physiological parameters of heart activities and analyze frequency spectrum distribution of respiratory signals and judge whether there is any interference from heart activities; E. Analyze the spectral energy envelop of respiratory waveform and find out the correct spectral peak, and then convert the frequency point corresponding to the spectral peak to the respiratory rate so as to obtain the value of current respiratory rate by integrating with the previous values of respiratory rate. The processing methods under this Invention can maximize the accuracy of results of respiratory rate and improve the stability of such result remarkably.

### TECHNICAL FIELD

This Invention relates to a respiratory signal processing method, and particularly to a conversion of respiratory time-domain waveform by means of a spectral method and analysis of distribution of waveform spectral energy for the purpose of anti-interference, acting as respiratory signal processing method for improving the stability and accuracy of respiratory detection.

### BACKGROUND ART

The existing respiratory measuring device often applies an impedance method-based measuring method to obtain the respiratory wave signals. During human respiratory movement, the muscle on the chest wall sees the alternating of muscular tension and relaxation; the thoracic cage deforms alternately; the electric impedance of thoracic cavity changes alternately accordingly—by detecting the subtle change of electric impedance, the respiratory change can be observed. During an actual respiratory measuring process, the electrodes fixed on the specific positions on body surface for body surface's electrocardiac signal detection is applied to send the hi-frequency carrier signal to human thoracic cavity so as to modulate the thoracic cavity's respiratory change-caused subtle impedance change to the hi-frequency carrier signal, and to obtain the simulative respiratory signal after amplification, detection and demodulation of such carrier signal by means of a series of circuits and finally figure out the respiratory rate as respiratory detection parameter and asphyxia warning information by means of respiratory algorithm on the basis of the digital respiratory signals derived from A/D conversion.

For eupnea, the respiratory rate of a newborn is at 30˜70 BPM (Beats Per Minute) and that of a adult is at 12˜30 BPM, but the range of respiratory detection shall be at 8˜120 BPM in general and at 150 BPM for some case when exceptions are taken into consideration. In such case, the frequency of respiratory wave corresponding to the detection range of such respiratory rate is at 0.125˜2.5 Hz.

The respiratory detecting method on the current market mostly includes the waveform method—for such method, the average value (i.e. baseline value) of waveform within a period time is applied to judge whether the current respiratory wave trends upward or downward, and the peak and trough of waveform is obtained by means of a method of extreme value. The effective peak or trough is judged according to some threshold conditions and then the waveform cycle is calculated according to the cycle of the effective peak or trough so as to obtain the respiratory rate, whereas the respiratory asphyxia is judged according to the average value of amplitude of waveform within a period of time.

Although the waveform method features the relatively visible computational process and less amount of operation, the actual clinic practice discovers that: when waveform is inordinate due to patient's restlessness, the accurate waveform cycle cannot be found out frequently and the miscalculation of respiratory rate is caused therefore; for the principle to detect the respiratory waveform by means of impedance method, the cardiovascular artifact will affect the results of such method due to the indispensable sharing of electrocardiac electrodes and the obtained respiratory waveform will be subject to the interference of ECG waveform more or less—in particular, the waveform cannot tell the cardiovascular artifact waveform correctly from the respiratory waveform in case of respiratory waveform's time domain mixed with very strong cardiovascular artifact and even completely submerged by the electrocardiac interference when patient is asleep or the ECG electrodes are positioned improperly or there is a brief respiratory obstruction; the baseline value figured out with the foregoing method cannot be upgraded quickly in case of the presence of waveform baseline shift and the respiratory rate will be on the low side due to the missing of waveforms. Moreover, the anti-interference ability of waveform method-based respiratory detection is weak and the cardiovascular artifact cannot be determined correctly.

### CONTENTS OF THE INVENTION

This Invention aims to overcome the shortcomings of the waveform method as an existing technology by providing a respiratory signal processing method with high accuracy and stability.

To solve the foregoing technical problem, this Invention is conceived as below: the conventional respiratory detection method is often to analyze the respiratory time-domain waveform, but this Invention analyzes the respiratory signal on the basis of frequency domain. The frequency domain distribution of waveform often has many features than cannot observed from the time domain and in particular, the respiratory waveform has a periodicity and its frequency domain will show the spectral peak with very strong energy intensity at the corresponding frequencies. Similarly, the respiratory waveform and jamming waveform that cannot identified in the time domain will show the spectral peak at different frequency points in the frequency domain. Hence, the time domain-based separation of respiratory waveform from jamming waveform is changed into the separation of different spectral peaks of frequency domain—the latter is more easily realizable and more accurate.

For achieving the objective above, this Invention adopts the following technical proposal:
A. Obtain the respiratory physiological signals by means of a specific respiratory circuit, AD collects and obtains the respiratory digital signals and applies the data preprocessing to such signals;
B. Convert frequency spectrum of the preprocessed respiratory signals and obtain the distribution of their frequency domain;
C. Judge whether there is asphyxia in accordance with the properties of respiratory signal's physiological parameters;
D. Introduce the physiological parameters of heart activities and analyze frequency spectrum distribution of respiratory signals and judge whether there is any interference from heart activities (i.e. cardiovascular artifact);
E. Analyze the spectral energy envelop of respiratory waveform and find out the correct spectral peak, and then convert the frequency point corresponding to the spectral peak to the respiratory rate so as to obtain the value of current respiratory rate by integrating with the previous values of respiratory rate.

The said band pass filter under Step A herein includes bi-directional IIR filter and multiplication-free elliptical IIR filter.

The said method to transform the frequency domain of respiratory signal under Step B herein includes the Fourier transform, wavelet transform and Hilbert transform.

The Fourier transform as one of the said transforming methods of frequency domain under Step B herein includes the fast Fourier transform (FFT) or Chirp Z-Transform (CZT).

After obtaining the frequency domain distribution of respiratory signal as described under Step C herein, the respiratory frequency domain-energy threshold and the conditions for respiratory waveform amplitude limit value of time domain will be set up firstly—it is judged as respiratory signal asphyxia when the maximum value of respiratory signal's spectral energy is less than the preset frequency domain-energy threshold line within a period of time while the time-domain waveform amplitude is less than the preset time-domain waveform threshold line. Such method judges the asphyxia of respiratory waveform by integrating the conditions of time domain and frequency domain so as to improve the accuracy of respiratory asphyxia detection.

The said introduced physiological parameter of heart activities under Step D herein include heart rate or pulse rate.

As to how to judge if the non-asphyxia waveform is subject to the cardiovascular artifact, the method is: obtain the maximum and sub-maximum values of respiratory spectral energy and then determine that the respiratory waveform is subject to the cardiovascular artifact to one degree or another if the corresponding respiratory rate falls into the set range of the current heart rate/pulse rate, and eliminate the frequency point while calculating the respiratory rate so as to enhance the anti-cardiovascular artifact ability of respiratory detection.

After the elimination of cardiovascular artifact, Step E herein will be implemented and include and such step shall also include as below:

a. Obtain the maximum value point of respiratory spectral energy and determine some spectral peaks.

b. Analyze the spectral peaks obtained under “Step A” herein and then determine a correct spectral peak according to the ratio of energy of each spectral peak, respiratory rate value corresponding to each spectral peak and ratio of historical respiratory rate values and convert the corresponding frequency point to the respiratory rate.

c. Obtain the weighted mean of the respiratory rate and historical respiratory rate calculated under “Step B’ herein and then get the current respiratory rate.

The foregoing technical proposal can eliminate the cardiovascular artifact more visibly and effectively and promote the reliability of asphyxia judgment; it is more easily for frequency domain to separate the jamming signal from the normal respiratory signal; the spectral peak can be judged with the help of historical respiratory rates—in such case, the anti-interference, accuracy and stability of respiratory can be enhanced.

### DESCRIPTION OF FIGURES

FIG. 1 shows the flow chart of this Invention;

FIG. 2 shows the respiratory data of normal respiration;

FIG. 3 shows the results of respiratory rate obtained from the normal respiratory data as processed by this Invention;

FIG. 4 shows the respiratory data of the respiration containing higher cardiovascular artifact;

FIG. 5 shows the results of respiratory rate obtained from the respiratory data containing higher cardiovascular artifact as processed by this Invention;

FIG. 6 shows the respiratory data when patient is restless;

FIG. 7 shows the results of respiratory rate obtained from the respiratory data as process by this Invention;

FIG. 8 shows the respiratory data of respiration containing higher interference;

FIG. 9 shows the results of respiratory rate obtained from the respiratory data containing higher interference as processed by this Invention.

### MODE OF CARRYING OUT THE INVENTION

This Invention will be described further in detail on the basis of attached diagrams and embodiments.

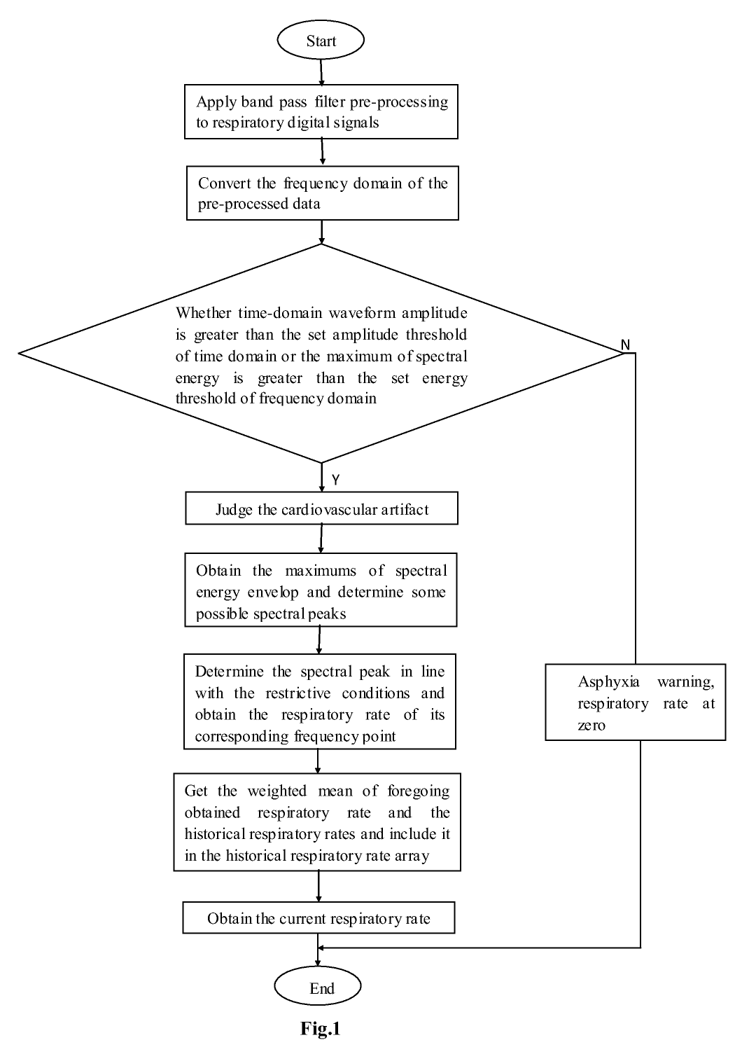

The monitoring facilities for respiratory signal measurement mostly include the master machine and slave machine—the slave machine obtains the respiratory data mostly through the hardware circuits whereas the master machine receives the data from the slave machine, displays the respiratory waveform, respiratory rate and asphyxia warning information. This Invention is mostly realized in the slave machine and the contents under this Invention can be moved to the master machine for such realizing in case of the slave machine's singlechip has an insufficient computing capacity. Shown as FIG. 1, the computing process of this method includes the main steps as below:
A. Apply the band pass filter data pre-processing to the respiratory digital signal.
B. Convert the frequency spectrum of the preprocessed respiratory signals and obtain the distribution of their frequency domain.
C. Set up the respiratory signal's frequency domain-energy threshold line and respiratory signal's time-domain waveform amplitude threshold line—it is judged as respiratory signal asphyxia when the maximum value of respiratory signal's spectral energy is less than the preset frequency domain-energy threshold line within a period of time while the time-domain waveform amplitude is less than the preset time-domain waveform threshold line.
D. Judge if the respiratory waveform is subject to cardiovascular artifact by means of the respiratory rate corresponding to the maximum and sub-maximum values of respiratory spectral energy and the current heart rate as the restrictive conditions (but not limited to).
E. Analyze the spectral energy envelop of respiratory waveform and find out the correct spectral peak, and then convert the frequency point corresponding to the spectral peak to the respiratory rate so as to obtain the value of current respiratory rate on the basis of the weighted mean of such respiratory rate and historical values of respiratory rate.

The band pass filter applied in the embodiment under Step A herein means the multiplication-free IIR filter (but not limited to).

The spectral transforming method involved in the embodiment under Step B herein means Chirp Z-Transform (but not limited to). The theoretical derivation includes as below:

It is known that x(n)(0≦n≦N−1) s the limited long sequence and its “Z” is changed to:

$$X ( z ) = {\sum }_{n = 0}^{N - 1} x ( n ) {z}^{- n} \qquad ( 1 )$$

To adapt to “z”, the value can be obtained along a route more generally than “z” plane, so the sampling is done on a section of spiral along the “z” plane with the equal subangles and these sample points zₖ of “z” includes:

zₖ=AW^{-k} k=0,1, . . . ,M−1 (2)

Wherein: A and W mean:

$$\{ \begin{matrix}A = {A}_{0} {e}^{j {θ}_{0}} \\ W = {W}_{0} {e}^{- j {φ}_{0}}\end{matrix} \qquad ( 3 )$$

So that the following can be obtained:

zₖ=A₀e^{jθ}^{₀}*W₀^{-k}e^{jφ}^{₀}^{k} k=0,1, . . . ,M−1 (4)

Wherein:

A₀ means the length of radius as a vector of the sample point;

θ₀ means the phase angle of the starting sample point z₀;

φ₀ means the difference of the angle between two neighboring sample points;

W₀ means the extension ratio of the spiral.

With zₖ from Equation (4) taken to Equation (1), the following result can be obtained:

$$X ( {z}_{k} ) = {\sum }_{n = 0}^{N - 1} x ( n ) {A}_{0} {W}_{0}^{- k} {e}^{j ( {θ}_{0} + k {φ}_{0} )} \\ k = 0 , 1 , … , M - 1 \qquad ( 5 )$$

After a series of transformations of arithmetic formula for CZT for the convenience of calculation, the following linear convolution can be obtained:

$$\begin{matrix}X ( {z}_{k} ) = {W}^{\frac{{k}^{2}}{2}} {\sum }_{n = 0}^{N - 1} g ( n ) h ( k - n ) = {W}^{\frac{{k}^{2}}{2}} [ g ( k ) * h ( k ) ] \\ k = 0 , 1 , … , M - 1 \\ Wherein \text{:} & ( 6 ) \\ \{ \begin{matrix}g ( n ) = x ( n ) {A}^{- n} {W}^{\frac{{n}^{2}}{2}} \\ h ( n ) = {W}^{- \frac{{n}^{2}}{2}}\end{matrix} \\ n = 0 , 1 , … , N - 1 & ( 7 )\end{matrix}$$

For the special case

$$M = N , {A}_{0} {e}^{j {θ}_{0}} = 1 , {W}_{0} = 1 , {ϕ}_{0} = \frac{2 π}{N} ,$$

each zₖ is distributed on the unit circle evenly at equal intervals—in such case, the Fourier transform of the sequence is evaluated. With θ₀ set, φ₀ and number of sample point can apply the spectral analysis to the signals within a certain frequency domain only.

In accordance with the feature that the frequency domain of respiratory wave detection falls in 0.125˜2.5 Hz, the above theoretical analysis shows that the embodiment herein determines the frequency domain of CZT at 0˜3 Hz.

To meet the requirement of ±1 BPM resolution for respiratory detection, the formula of resolution for CZT can be obtained as below:

$$\frac{( {f}_{2} - {f}_{1} ) * 60}{N} = 1$$

wherein: f₁=0 Hz and f₂=3 Hz, so as to work out that the number of dot of CZT is at 256. The sampling rate of frequency domain transform for respiration of the embodiment herein is fₛ=25 Hz. To ensure that the data for a frequency domain transform include at least two respiratory cycles, the embodiment herein determines CZT's number of dot at N=512 with respect to the low-frequency signal of 6 BPM and 20 S of two waveform cycles.

After the CZT of some single-frequency signal, the spectral analysis will be performed and there will be a maximum peak value of spectrum at such frequency; after the CZT of the signal derived from the signal superposition of multiple frequencies, the spectral analysis will be performed and the spectral energy at each frequency will have a local maximum—out of all local maximums, the frequency corresponding to the top local maximum shows that the signal of such frequency is the strongest during superposition within the time domain.

In accordance with the foregoing theoretical basis, the respiratory signal's frequency domain-energy threshold line and respiratory signal's time-domain waveform amplitude threshold line are set—it is judged as respiratory signal asphyxia when the maximum value of respiratory signal's spectral energy is less than the preset frequency domain-energy threshold line within a period of time while the time-domain waveform amplitude is less than the preset time-domain waveform threshold line.

The cases with respiratory waveform will be analyzed thereafter on the basis of the steps as below:
Firstly, obtain the respiratory rates corresponding to the maximum and sub-maximum spectral energy values—it shows that there is very strong cardiovascular artifact in respiration and even the normal respiratory waveform is submerged therefore if the said respiratory rate corresponding to the said maximum of spectral energy falls in the range of ±r of heart rate, then it is suggested giving the cardiovascular artifact warning to prompt it may be necessary to readjust the electrode position so as to obtain a better respiratory waveform; it shows that the intensity of cardiovascular artifact imposed to the respiratory waveform is not serious and no warning will be given, with the related frequency point eliminated, if the submaximum value corresponding to the spectral energy falls in the range of ±r of heart rate whereas the said maximum of spectral energy does not falls in the range of ±r of heart rate.
Secondly, search for all local maximums in spectral energy envelop of respiratory waveform and determine some possible spectral peaks.

The spectral peaks obtained hereinabove will be analyzed and eliminated according to the ratio of energy of spectral peaks, respiratory rate corresponding to spectral peaks and ratio of historical respiratory rates so as to determine a correct spectral peak. In principle, the true spectral domain distribution of respiratory waveform should fall on the maximum, submaximum and the third maximum.

The weighted mean is applied to the respiratory rates and historical respiratory rates corresponding to the obtained correct spectral peak so as to find out the current respiratory rate for further enhanced stability of computing of respiratory rate.

To describe the spectral method-based computing of respiratory more visibly, examples will be taken from the normal respiratory waveform derived from the actual clinic practice, the cardiovascular artifact-contained respiratory waveform, the restlessness interference-contained respiratory waveform and the respiration contained very high interference so as to demonstrate of the accuracy of spectral method-based computation of respiratory rate in processing different kinds of respiratory data.

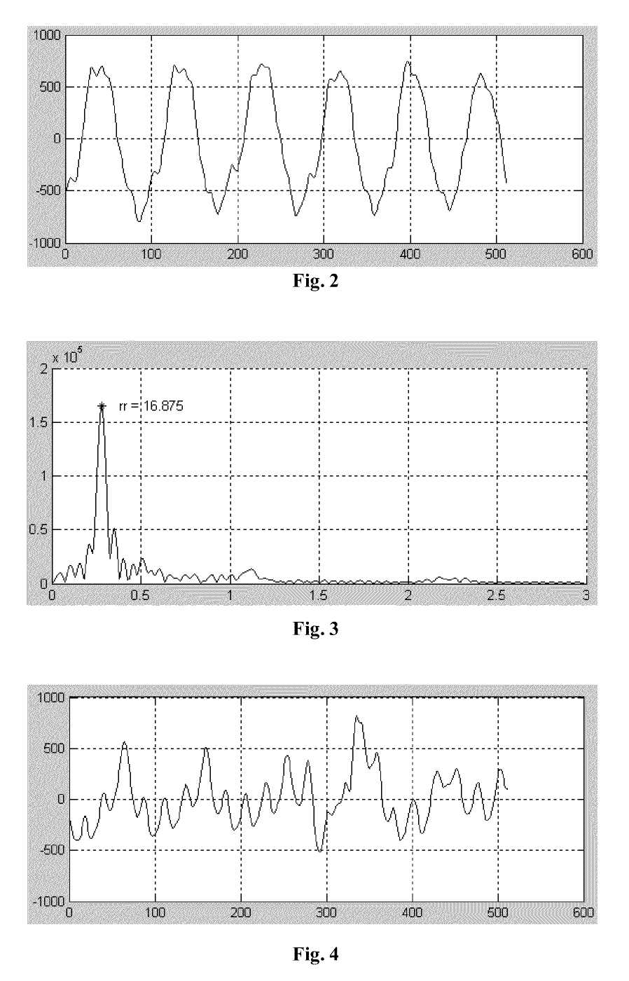

As to the normal respiratory waveform as shown in FIG. 2, the respiratory waveform is regular and free of jamming, and shown as FIG. 3, the cyclic waveform features highly concentrated energy distribution in frequency domain. The spectral energy at the asterisk shown in FIG. 3 is the top value among all local maximums of spectral energy envelops, and the respiratory rate of the respiratory waveform described in FIG. 2 will be obtained after converting the frequency point corresponding to such spectral energy to the respiratory rate per minute.

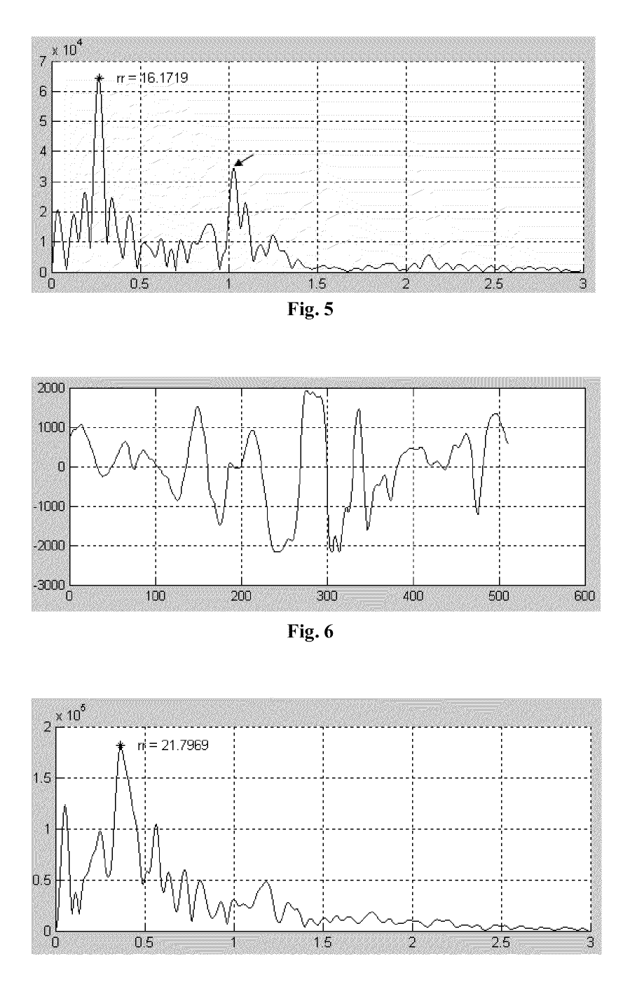

As to the case that respiratory wave contains higher cardiovascular artifact as shown in FIG. 4, there will be two bigger peaks in its frequency spectrum as computed by the spectral method—the frequency corresponding to the top one out of these two bigger peaks means the frequency of respiratory wave whereas the frequency corresponding to the other peak means the frequency of palpitation, with the respiratory rate marked with an asterisk and the spectral peak corresponding to cardiovascular artifact marked with an arrow in FIG. 5. The jamming caused by the electrocardiac waveform and heart activities is more discrete in frequency domain than in time domain and FIG. 5 shows the distinctive advantage in computing the respiratory rate of the cardiovascular artifact-contained respiration vividly.

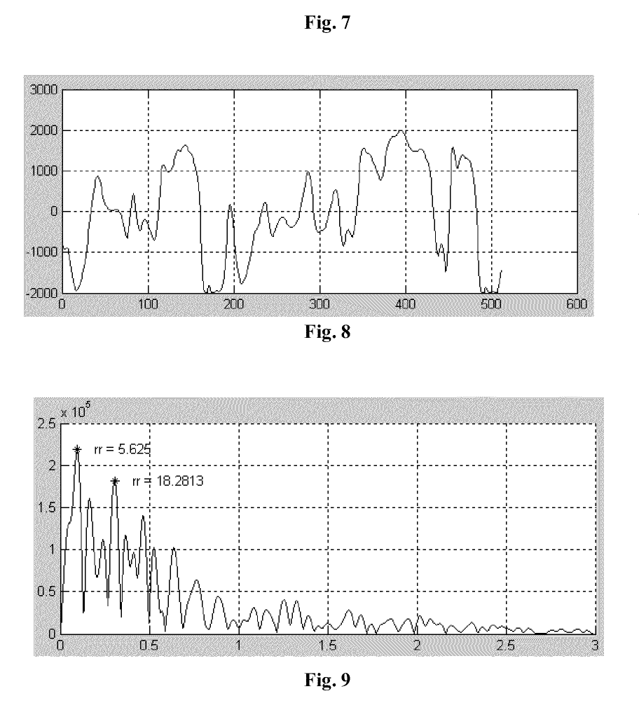

As to the case that the respiratory wave contains restlessness interference as described in FIG. 6, the spectral method can work out the respiratory rate very easily on the basis of frequency domain although the respiratory waveform is relatively disordered and its shape is irregular, with the respiratory rate as described in FIG. 7, whereas the waveform method features a bigger calculation error for such respiratory wave.

As to the case that the respiratory wave contains very high interference as described in FIG. 8, the respiratory waveform is very inordinate and the shape is very irregular. For the spectral method-based calculation, the spectral energy ratio of spectral energy's maximum and submaximum is equivalent, but the respiratory rate corresponding to the energy maximum is very low; the spectral peak corresponding to the submaximum is selected as the current correct spectral peak according to the proportional relation with the historical respiratory rates—such case is relatively true, with the result shown in FIG. 9.

The foregoing example shows: the spectral method can figure out the respiratory rate correctly when the normal respiration or the intensity of respiration-contained jamming is weaker than the respiratory signal; the respiratory rate can be worked out relatively correctly by means of the restrictive conditions provided under this Invention when the respiration contains restlessness jamming or other higher interferences. Furthermore, this Invention also provides the condition to judge the current respiratory rate with the data of the historical respiratory rates, so that the computational solution of respiratory rate is more accurate, stable and reliable.

1. A respiratory signal processing method, including the steps as below:
A. Obtain the respiratory physiological signals by means of a specific respiratory circuit, AD collects and obtains the respiratory digital signals and applies the data preprocessing to such signals;
B. Convert frequency spectrum of the preprocessed respiratory signals and obtain the distribution of their frequency domain;
C. Judge whether there is asphyxia in accordance with the properties of respiratory signal's physiological parameters;
D. Introduce the physiological parameters of heart activities and analyze frequency spectrum distribution of respiratory signals and judge whether there is any interference from heart activities (i.e. cardiovascular artifact);
E. Analyze the spectral energy envelop of respiratory waveform and find out the correct spectral peak, and then convert the frequency point corresponding to the spectral peak to the respiratory rate so as to obtain the value of current respiratory rate by integrating with the previous values of respiratory rate.

2. In accordance with the respiratory signal processing method described under claim 1 herein and its feature is shown as below: the said data preprocessing under the Step A means the band pass filter applied to the respiratory signals.

3. In accordance with the respiratory signal processing method described under claim 2 herein and its feature is shown as below: the said band pass filtering means the bi-directional zero phase IIR filter.

4. In accordance with the respiratory signal processing method described under claim 2 herein and its feature is shown as below: the said band pass filter means the multiplication-free elliptical IIR filter.

5. In accordance with the respiratory signal processing method described under claim 1 herein and its feature is shown as below: the said method of frequency spectrum conversion means the Fourier transform.

6. In accordance with the respiratory signal processing method described under claim 1 herein and its feature is shown as below: the said method of frequency spectrum conversion means the wavelet transform.

7. In accordance with the respiratory signal processing method described under claim 1 herein and its feature is shown as below: the said method of frequency spectrum conversion means the Hilbert transform.

8. In accordance with the respiratory signal processing method described under claim 5 herein and its feature is shown as below: the said Fourier transform means the fast Fourier transform (FFT).

9. In accordance with the respiratory signal processing method described under claim 5 herein and its feature is shown as below: the said Fourier transform means the Chirp Z-Transform (CZT).

10. In accordance with the respiratory signal processing method described under claim 1 herein and its feature is shown as below: the said asphyxia judging method means setting up the respiratory signal's frequency domain-energy threshold line and respiratory signal's time-domain waveform amplitude threshold line—it is judged as respiratory signal asphyxia when the maximum value of respiratory signal's spectral energy is less than the preset frequency domain-energy threshold line within a period of time while the time-domain waveform amplitude is less than the preset time-domain waveform threshold line.

11. In accordance with the respiratory signal processing method described under claim 1 herein and its feature is shown as below: the said introduced heart activity parameter means the value of heart rate.

12. In accordance with the respiratory signal processing method described under claim 1 herein and its feature is shown as below: the said introduced heart activity parameter means the value of pulse rate.

13. In accordance with the respiratory signal processing method described under claim 1 herein and its feature is shown as below: the said judging method of cardiovascular artifact means obtaining the maximum value and sub-maximum value of respiratory spectral energy—the respiratory waveform is judged to be interfered by the palpitation interference to one degree or another if the corresponding value of respiratory rate falls into the current set range of heart rat and/or pulse rate values.

14. In accordance with the respiratory signal processing method described under claim 1 herein and its feature is shown as below: the said analyzing method of respiratory wave frequency domain-energy envelop means obtaining the maximum value point of respiratory SIG's spectral energy and determining some spectral peaks.

15. In accordance with the respiratory signal processing method described under claim 1 herein and its feature is shown as below: the said finding out of the correct spectral peak means analyzing the obtained spectral peak so as to determine a correct spectral peak according to the ratio of energy of each spectral peak, respiratory rate value corresponding to each spectral peak and ratio of historical respiratory rate values and to convert the corresponding frequency point to the respiratory rate.

16. In accordance with the respiratory signal processing method described under claim 1 herein and its feature is shown as below: the said method to obtain the current respiratory rate value means analyzing the obtained spectral peaks so as to determine a accurate spectral peak according to the ratio of energy of each spectral peak, respiratory rate value corresponding to each spectral peak and ratio of historical respiratory rate values, to convert the corresponding frequency point to the respiratory rate and calculate the weighted mean of the obtained respiratory rate values and historical respiratory rate values.
